(19) United States
(12) Patent Application Publication
(10) Pub. No.: US 2007/0196884 A1
(43) Pub. Date: Aug. 23, 2007

(54) Method For The Detection Of Coliforms And In Particular Escherichia Coli

(75) Inventors: Sergio Bodini, Magenta (IT); Francesca Santori, Terni (IT)

Correspondence Address: NIXON PEABODY, LLP, 401 9TH STREET, NW, SUITE 900, WASHINGTON, DC, 20004-2128, US

(73) Assignee: ISRIM S. CONS. A.R.L., Terni (IT)

(21) Appl. No.: 10/566,445

(22) PCT Filed: Jul. 27, 2004
(86) PCT No.: PCT/EP04/51626

§ 371 Date: Aug. 21, 2006

(30) Foreign Application Priority Data
Jul. 31, 2003 (IT) TR2003A000002

(51) Int. Cl. C12Q 1/34; C12Q 1/04

(52) U.S. Cl. 435/18; 435/34

(57) ABSTRACT

The invention is relative to a novel method for the detection of coliforms, which is based on the use of a mixture of amino acids to promote the expression of inducible enzymes, in absence of cell growth. This enzyme-inducing solution can be used for the rapid detection of the enzymes β-glucuronidase and/or β-galactosidase in a very small number of E. coli and/or total coliforms.

### TECHNICAL FIELD

The present invention relates to a method for detecting coliforms which employs an enzyme-inducing solution containing one or more amino acids, capable to promote the expression of inducible enzymes in absence of cell growth. This method can be used for the rapid detection of a very small number of E. coli and/or total coliforms, by measuring enzymatic activity of the enzymes β-glucuronidase and β-galactosidase.

### BACKGROUND ART

Many rapid methods for the identification/detection of microorganisms are based on the presence of enzymes involved in specific metabolic activities. These enzymes can be constitutive, which means always expressed, or inducible, namely present only in determined metabolic conditions. That is the case of the enzymes β-glucuronidase and β-galactosidase expressed in coliform bacteria in presence of a specific inducer.

Coliform bacteria are “indicator microbes” of the presence of traces of human sewage. They are commonly divided in total coliforms and faecal coliforms, including the former also coliforms found in surface soil and water, and the latter, the most common member being E. coli, only gram-negative thermo resistant bacteria, naturally inhabiting both human and animal gastrointestinal tract, causing, thus, urogenital infections and diarrhea.

Total coliforms are characterized by the possession of the gene coding for the enzyme β-galactosidase, responsible for the hydrolysis of lactose to galactose and glucose. The determination of β-galactosidase can be achieved by means of chromogenic and/or fluorogenic substrates.

β-glucuronidase is an enzyme which catalyzes the hydrolysis of β-D-glucopyranosiduronic acids into the correspondent aglycons and D-glucuronic acid; like β-galactosidase, the activity is measured by using different chromogenic and/or fluorogenic substrates. It is present in the 94-96% [Acta Pathol. Microbiol. Scand. Sect. B, 84:245(1976)] of different types of E. coli. It is therefore specifically used for the detection of E. coli.

The production of β-galactosidase can be enhanced by using the “natural” inducer lactose or non-metabolizable analogues, like methyl-β-D-thiogalactopyranoside (MTG) and isopropyl-β-D-thiogalactopyranoside (IPTG) that, not releasing the glucosidic part, do not cause the interruption of the enzymatic synthesis. The synthesis of β-glucuronidase may be similarly stimulated by methyl-β-D-glucuronide or isopropyl-β-D-glucuronide. Literate data show that, up to today, the methods for detecting the expression of those enzymes are based on the enzymatic induction achieved by means of specific growth media, where the production of β-galactosidase/β-glucuronidase is promoted through a process of cell duplication. Carbon sources (ike lactose), nitrogen sources, vitamins and mineral salts are usually contained in common culture media, in order to assist the desired growth.

Berg and Fiksdal disclose (patents U.S. Pat. Nos. 55,188,894, 5,292,644, EP0574977 and WO8904372) the use of a nutritive growth media, selective for coliforms, containing lactose as a production agent for β-galactosidase, yet enriched with the fluorogenic enzyme substrate methylumbelliferyl-β-D-galactoside (MUGal), providing thus a fluorimetric response after not more than 8 hours of incubation. The evolution of this process leads to the development of a kit for rapid detection of coliforms known as Colifs™ (patents U.S. Pat. Nos. 55,188,894, 5,292,644, EP0574977 and WO8904372). Using this system, Tryland et al. [Wat. Sci. Tech., Vol.43 No°12 217-220 (2001)] and Fanleitner et al. [Lett. Appl. Microbiol. Vol.33 Iss.3 246-250 (2001)] verify, respectively, a highly significant relationship between β-galactosidase activity and coliforms count, and between the rate of β-glucrronidase hydrolysis and E. coli concentration. This method, compared to the method of the current invention, presents the following disadvantages:
1. the cell duplication introduces another variable influencing the fnal result; besides the enzyme produced at the single cell level, also the parameter of the final cell count must be taken into account;
2. the detection time, especially when a number of cells <1000 is present in the sample, is strongly conditioned from the need to quantitate in the exponential phase; time proposed from the Applicant is shorter.

Edberg (U.S. Pat. No. 4,925,789, 1990) discloses the concept of “nutrient-indicator”. The growth medium includes, as a primary carbon source, a specific nutrient, modified by attaching a sample-altering moiety thereto, which only the target microbe can metabolize. The moiety is activated to alter the sample only if the nutrient is metabolized by the target microbe, defining therefore its presence/absence. Edberg specifically detects E. coi, using a chromogenic or fluorogenic substrate, selected among o-nitrophenyl-β-D-glucuronide (ONPG, yellow), β-naphtalamide-β-D-glucuronide (purple), α-naphtol-β-D-glucuronide (red), methylumbelliferyl-β-D-glucuronide (MUGlu, fluorescent). He also tests combinations among them for their ability to determine simultaneously E. coli and total coliforms, by shuffling different substrates for β-glucuronidase (E. coli) and for β-galactosidase (coliforms). The incubation times vary between 18 and 22 hours. The product Coliler™ and the MMO/MUG test, approved from the U.S. Environmental Protection Agency (USEPA), where ONPG is used for total coliforms and MUGlu for E. coli, are based on the general principles disclosed by Edberg. This procedure requires at least 18 hours to complete. Time proposed from the Applicant is shorter.

Roth et al. (U.S. Pat. No. 5,210,022, 1993) disclose test media and chromogenic compounds for identifying and differentiating E. coli and general coliforms. The test medium is formed by combining a nutrient base medium with a chromogenic substrate for β-galactosidase, which causes the formation of an insoluble precipitate of a first colour upon reacting with the enzyme β-gal, and also a chromogenic substrate for β-glucuronidase, capable to generate an insoluble precipitate of a second colour contrasting with the first colour upon reacting with the enzyme GUD. From this disclosure takes place the commercial product Colichrome 2™. The method requires 24-48 hours incubation. Time proposed from the Applicant is shorter.

Brenner et al. [Appl. Environ. Microbiol. 1993 Nov; 59(11):3534-44] provide the improved medium MIagar, incorporating methylumbelliferyl-β-D-galactoside (MUGal) as the β-galactosidase substrate to identify total coliforms, and indoxyl-β-D-glucuronide (Ibdg) for detecting E. coli through the β-glucuronidase activity (U.S. Pat. No. 6,063,590. 2000). This method, also intended as a cell growth procedure, takes at least 20 hours. Time proposed from the Applicant is shorter.

In an article Van Poucke and Nelis [Appl. Environ. Microbiol. Vol.63 No°2 771-774 (1997)] describe a chemiluminometric test for the detection of total coliforms and E. coli in a liquid mediumn. The new medium Colicultm allows to reduce detection time to 6 hours for total coliforms, being Galacton-Plus enzymatic substrate and inducer, and 9 hours for E. coli, being Glucuron substrate and inducer (U.S. Pat. No. 5,861,270, 1999). This method, like already explained when treating the procedure disclosed by Berg and Fiksdal, suffers disadvantages due to cell duplication and long detection time (6-9 hours), because of the need to quantitate in the exponential phase of cell growth. The method proposed from the Applicant provides a shorter detection time and cell growth is not required.

Grant (U.S. Pat. No. 5,849,515, 1998) discloses a culture medium which permits simultaneous detection of total coliforms and E. coli, by using a selective β-glucuronidase substrate, like the salt cyclohexylammonium 5-bromo-4cbloro-3-indolyl-β-D-glucuronate (X-Gluc), and a highlighting dye, like triphenyltetrazolium chloride, for enhancing visual differentiation in the detection of β-galactosidase. Originated from this disclosure is the product m-Coliblue24. In the composition of the growth medium, lactose is both carbon source and enzymatic inducer, and some essential amino acids are contrast promoters for increasing the formation of β-glucuronidase by E coli. Even though the importance of some amino acids is evidenced, the spirit of the work is still definitely directed to a cell growth system leading to a cfu (colony forming units) count. The test lasts therefore 36-48 hr.

The Applicant is able, in any case, to raise at the maximum rate, the enzymatic synthesis at the single cell level, independently from the environmental conditions from which the bacteria happen to be sampled; it is actually impossible a definition a priori of “optimal induction”, being the enzymatic profile strongly dependent from the outer environment.

In conclusion:
1. methods based on cells count on Petri dishes imply analysis times exceeding 12 hours;
2. methods based on a direct analysis in liquid medium also need to quantitate in the exponential phase of cell growth, resulting in slow detection times; another variable, concerning the cell growth, must be taken into account when defining a linear correlation between the enzymatic activity and the original number of cells;
3. methods based on the presumption that, in determinate environmental circumstances, the enzymatic activity of coliform cells is natually induced, limit the universal feasibility of the process, subject the results to false negatives, and turn out to be strictly dependent from the level of enzymatic induction present at the moment of sampling.

The current invention discloses a sensitive and rapid method for the detection of coliforms, performed in absence of cell growth, which overcomes the above-mentioned deficiencies and disadvantages.

The method hereby described is based on the simple observation that the amino acids, singularly or in a mixture, are capable of sensibly incrementing the expression of β-galactosidase and β-glucuronidase in the single coliform cell, through a process not requiring cell duplication; this occurrence allows to achieve a detectable amount of enzymes, even when dealing with a very poor number of cells.

### DISCLOSURE OF INVENTION

The object of the current invention is an induction solution that, in absence of cell growth, is capable to induce the expression of inducible enzymes in target microbes; this solution can be therefore utilized for the rapid detection of E. coli and total coliforms by inducing selective enzymes β-glucuronidase and β-galactosidase. The induction solution includes:
1. at least one amino acid, and preferably a mixture of amino acids, being in a concentration not to allow a detectable cell growth within a time between 0 and 120 minutes;
2. a buffer system, like for instance sodium hydrogen phosphate (Na₂HPO₄) and potassium dihydrogen phosphate (KH₂PO₄), preferably, but not necessarily, providing a pH between 6.0 and 7.5 and comprising sodium chloride (NaCl), preferably, but not necessarily at 0.01% (w/v);
3. bivalent ions and, more specifically, the magnesium Mg⁺⁺, in a concentration preferably 0.5 mM;
4. an enzymatic inducer (i.e. isopropyl-β-D-thiogalactopyranoside for β-galactosidase, concentration being preferably 0.2 mM, methyl-β-D-glucuronide for β-glucuronidase, concentration being preferably 2 mM, and relative mixtures).

According to the invention, the amino acids are essential to achieve the enzymatic induction; the mixture of amino acids can preferably comprise 20 amino acids or only some of them, being their concentration preferably comprised between 0.01 mM and 0.05 mM each.

Still this mixture can comprise at least the following amino acids:
tryptophan W;
at least one between methionine M and threonine T;
isoleucine I and leucine L.

Yet still this mixture preferably comprises the 20 natural amino acids in a levorotatory form (alanine A, cysteine C, aspartic acid D, glutamic acid E, phenylalanine F, glycine G, histidine H, isoleucine I, lysine K, leucine L, methionine M, asparagine N, proline P, glutamine Q, arginine R, serine S, threonine T, valine V, tryptophan W, tyrosine Y).

Another aspect of the invention concerns a method for detection of microorganisms, preferably in water samples or any other sample in which the microbes can be extracted, for analytical purposes, in liquid environment.

Yet another aspect of the invention is a method allowing the determination of the concentration of total coliforms or E. coli in waste water, surface water, bathing water, fresh water, sea water and urban water. Solid samples like food, soil, etc., requiring a microbiological investigation, may be analysed with this method after opportune extaction in liquid phase.

According to necessity the sample can be:
directly added to the induction solution;
filtered to concentrate bacteria on a membrane filter with a pore size not superior to 0.45 μm, then the membrane filter and the bacteria concentrated thereon is immersed in a minimum volume of induction solution, enough to cover up the retained material;
extracted with suitable liquid medium, filtered to concentrate bacteria on a membrane filter with a pore size not superior to 0.45 μm, then the membrane filter and the bacteria concentrated thereon are immersed in a minimum volume of induction solution, enough to cover up the retained material.

The induction step is performed between 30° C. and 40° C. for total coliforms and between 30° C. and 50° C. for faecal colifonns or E. coli, for a period of time not longer than 120 minutes (after 2 hours of incubation, significant cell growth is observed). The detection step is performed by using a fluorimetric assay, known in literature, consisting in an extracellular enzymatic reaction of target enzymes β-galactosidase and β-glucuronidase with the relative fluorescent substrates methylumbeuliferyl-β-D-galactoside (MUGal) and methylumbelhferyl-β-D-glucuromnide (MUGlu). The hydrolysis reaction starts with the addition of the enzymatic substrate and organic solvent. The reaction is stopped by adding sodium or potassium hydroxide, which also amplify the fluorescence signal. There are no time limits for the hydrolysis, usually lasting from 5 to 120 minutes and preferably, but not necessarily, lasting 15 minutes. The lowest amount of cells detectable with the present method is about 10 cells/sample. Hydrolysis is preferably shorter than 120 minutes, more preferably shorter than 60 minutes, still more preferably shorter than 30 minutes and most preferably shorter than 15 minutes. In any case the reaction time can be adjusted according to the number of cells. The method is so sensitive that it can detect down to 10 cells per sample and although exceeding, in such an eventuality, 20 minutes duration, will still be time-restrained (typically less than 3 hours) if compared to other known methods.

According to a preferred embodiment of the invention, the method for the detection of total coliforms is based on the use of the following reagents in agreement with the procedures hereby described; in a more preferred embodiment, the reagents can be assembled in kit form:
solution of enzymatic induction (Reagent A): Reagent A comprises a water solution, pH between 2 and 10, including: a system buffer, like, for instance, sodium hydrogen phosphate (Na₂HPO₄), potassium dihydrogen phosphate (KH₂PO₄), magnesium sulphate heptahydrate (MgSO₄.7H2O), preferably but not necessarily at pH 7,2 (β-galactosidase) and pH 6,5 (β-glucuronidase); bivalent ions and, more specifically, the magnesium Mg⁺⁺, which are essential at a concentration, preferably but not necessarily, 0.5 mM; at least an amino acid, preferably but not necessarily, tryptophan W, in a concentration up to 80 mM; more preferably but not necessarily another amino acid, chosen between methionine M and tbreonine T, in a concentration up to 80 mM and, still more preferably but not necessarily, also isoleucine I and leucine L, in a concentration up to 80 mM; in general the use of the all natural 20 amino acids, promoters of the induction, is preferable but not necessary, at a concentration, preferably but not necessarily, 0.02 mM each; an inducer of β-galactosidase or β-glucuronidase, like, for instance, isopropyl-β-D-thiogalactopyranoside or methyl-β-D-glucuronide and relative mixtures; a selective agent, acting as a membrane permeabilizer, like, for instance, sodium dodecyl sulphate; sodium chloride NaCl, preferably but not necessarily, at 0.01% (w/v); the solution may be sterilized, for instance, by filtering through a membrane filter with a pore size not superior to 0.45 μm, and can also eventually be in a lyophilized form;
solution of enzymatic substrate (Reagent B): Reagent B comprises a solution of methylumbelliferyl-β-D-galactoside (MUGal) dissolved in dimethylsulifoxide and phosphate buffer, or a solution of methylumbelliferyl-β-D-glucuronide (MUGlu) dissolved in a solution of phosphate buffer/triton-x 99/1, or a mixture of both of them, preferably but not necessarily, characterized from the absence of 4-methylumberiferone (MU), opportunely separated, for example, by passage on anionic column (i.e. resin Amberlite IRA-410 Sigma), to reduce the natural presence of 4-methylumbelliferone; the reagent can also eventually be in a lyophilized form;
solution of fluorescence amplification (Reagent C): Reagent C comprises a sodium hydroxide (NaOH) solution in water, or any other base with the purpose of sensibly enhancing the natural fluorescence of 4-methylumbelliferone (up to 4 times), effect feasible at pH 11-12;
solvent: the organic solvent (for example chloroform or other suitable solvent) causes the rupture of the bacterial membrane and, consequently, the cell lysis.

Moreover Reagent A and Reagent B can be in a lyophilized form and added to the original sample, without any sample pretreatment, hence restoring an ideal environment of enzymatic induction and/or enzymatic hydrolysis, like hereby described.

The procedure according to the invention preferably comprises the following steps:

1. Sample Preparation:

the sample can be analyzed directly, without any pre-treatment, by adding it directly to Reagent A; solid or semisolid samples can be extracted with a required volume of suitable buffer (preferably but not necessarily, a physiological solution or the formerly described phosphate buffer) and shortly processed like non pre-treated samples; alternatively, it is possible to separate the bacterial cells from the water sample by physical treatment, like filtering to concentrate bacteria on a membrane filter with a pore size not superior to 0.45 μm.
   2. Enzyme Induction
as previously described, the sample can be:
   directly added to Reagent A;
   integrated with lyophilized Reagent A or lyophilized Reagent A+Reagent B, hence fitting the state of enzymatic induction;
   filtered to concentrate bacteria on a membrane filter and the filter immersed in a minimum volume of Reagent A, enough to cover up the retained material; in all the embodiments the system “sample-Reagent A” is incubated between 30° C. and 40° C. for total coliforms, and between 30° C. and 50° C. for faecal coliforms or E. coli, for a period of time not longer than 120 minutes, for example at least 5 minutes, like aforesaid;
      3. Expression of the Activity of the Induced Enzyme through Lysis of the Cell Membrane:
prior to, throughout or subsequent to the induction phase, Reagent B is added to the system “sample-Reagent A” (preferably but not necessarily 50 μl/ml Reagent B and instantly following the induction period); Reagent B contains the fluorogenic substrate; after induction, the rupture of the bacterial membrane and, consequently, the cell lysis, is achieved by adding an organic solvent, for example chloroform or other suitable solvent; this allows the induced enzyme to exit the cell; the hydrolysis of the fluorogenic substrate takes place, with discharge of the fluorescent cleavage product; after vortexing, the sample is incubated at a temperature not higher than 50° C., for a time that can typically last between 5 and 15 minutes, for example 10 minutes; at the end of this period, the reaction is stopped by adding Reagent C up to pH 11-12.
   4. Measurement:
the resulting solution is transferred to a quartz cuvette, and then placed in the fluorimeter provided with heating block; the excitation wavelength is set between 330 and 390 nm, the emission wavelength is set between 410 and 470 nm with slit widths between 2.5 and 20 mn; a blank reading needs to be performed by simulating the sample procedure, except for the inducing agents, in order to account for contaminating particles and background fluorescence; the control value must be subtracted from the sample result; in case of absence of interfering substances, it is also possible to achieve a control value just by simulating the sample analysis, except for the addition of the cells; operating on the field, having thus to deal with potential filter-retained interferents, the blank can be performed by omitting the step of cell lysis.
   5. Evaluation of Results:
a positive test result is defined as a statistically significant increase, when compared with control, in the fluorescence measurement, expressed in instrument-dependent arbitrary units; test positivity can be both qualitatively and quantitatively assessed; the use of a qualitative approach allows to differentiate total coliforms from faecal coliforms, according to the induction temperature, and faecal coliforms from E. coli, by means of their specific enzyme; a quantitative evaluation requires the elaboration of a fluorescence intensity calibration curve achieved by measuring samples containing known concentrations of target microbes (total coliforms, faecal coliforms or E. coli); a linear relation between fluorescence intensity and bacterial concentration can then be determined, allowing to calculate the number of cells contained in unknown samples; the calibration line is strictly dependent on the specific operating conditions; in order to provide reliable results, every little variation of parameters shall consequently be supported by the calculation of a fresh regression line.

Also contemplated as falling within the scope of the invention is an analysis kit comprising:
Reagent A, Reagent B, Reagent C;
instructions for use;
suitable instrumentation and solvents.

The advantages of the new method include the following:

1. The Dramatic Reduction of Detection Time:

the analysis is preferably shorter than 120 minutes, more preferably shorter than 60 minutes, still more preferably shorter than 30 minutes and most preferably shorter than 15 minutes; in any case, the reaction time can be adjusted according to the number of cells; the method is so sensitive that it can detect down to 10 cells per sample and, although exceeding, in such an eventuality, 20 minutes duration, it will still be time-restrained (typically less than 3 hours) if compared to other known methods, owing to the use of a new induction medium, able to stimulate, in absence of numerical cell growth, the production of detectable quantities of β-galactosidase and β-glucuronidase, even in samples containing a very small number of cells;
   2. The Original Use of a Mixtore of Amino Acids (“Induction Promoter”):
the mixture accounts for the aforementioned inductive properties, characteristic of the medium;
   3. The Absence of Nutritive Substances in the Induction Medium Preventing Unwanted Signal Increase Due to Cell Growth:
there is no need to wait for the exponential phase and, therefore, the measured enzymatic activity trs to be directly associated with the number of microorganisms originally contained in the sample; the variable connected to cell duplication shall not be considered, and a potential overestimation of the results during the fluorimetrc measurement is thus circumvented;
   4. The Presence of ion Magnesium Mg₊₊:
by acting like cofactors for β-galactosidase and β-glucuronidase, ions magnesium are absolutely indispensable both in the enzymes induction and hydrolysis;
   5. The Further Increase of β-Galactosidase Activity as a Consequence of the Use of Higher Substrate Concentration, Achieved Thanks to the Solubillzation in Dimeftylsulfoxide (Dmso) and Potential Purification on Anionic Column:
because of Dmso, the solubility of methylumbelliferyl-β-D-galactoside (MUGal) increases by 100 times; anionic resin helps remove free methylum-belliferone contaminating commercial methylumbelliferyl-β-D-galactoside (MUGal), resulting thus in a remarkable reduction of background fluorescence;
   6. Improved Detectability of the Fluorophore Due to Extracellular Enzyme Hydrolysis:
the targeted enzymes reside inside the cell; chloroform leads to cell lysis and discharge of those intracellular induced enzymes; the enzymatic reaction with relative substrates, being everything in solution, is thus accelerated, and the free fluorophore can easily be determined spectrofluorimetrically.

The use of an “induction solution” for the detection of E. coli and total coliforms, object of the current invention, includes the following advantages:
1. the reduction in detection time (from 10 to 120 minutes), due to induction not being associated with cell growth, together with the high sensitivity of the analysis, due to induction being directly promoted from amino acids, which stimulate the production of a detectable amount of enzyme, even in a very small number of cells;
2. the precision of the result and the linear correlation between induced enzymatic activity and number of cells, due to absence of cell proliferation; the cell growth would actually introduce another variable in the correlation between the enzymatic activity and the original number of cells.

The induction solution, object of the present invention, was used to perform the analyses described in the following examples; a fluorimetrc assay, based on the extracellular reaction of the enzymes with relative substrates methylumbelliferyl-β-D-galactoside (MUGal) and methylumbelliferyl-β-D-glucuronide (MUGlu), was selected to measure β-galactosidase and β-glucuronidase activity.

These and other objects of the invention will become more readily apparent from the following detailed description of several preferred embodiments of the invention. However, the examples set forth herein are in no way intended to limit the scope of the invention.

### MODE FOR THE INVENTION

### Example 1

Induction of β-Galactosidase Activity on E. coli and Enterococcus Faecalis and Cell Count When Positive.

Sample Preparation

Different samples in physiological solution (sodium chloride NaCl, 0.85% in water) containing E. coli cells (ATCC 25922) and Enterococcus faecalis cells (ATCC 29212) were variously diluted. For each sample, the cell number was verified by plate count method.

Enzyme Induction

33 μl of sample were added to 2315 μl of filter-sterilized (0.45 μm) induction solution, at pH 7.2, comprising sodium hydrogen phosphate (Na₂HPO₄) 47.7 mM, potassium dihydrogen phosphate (KH₂PO₄) 22 mM, magnesium sulphate heptahydrate (MgSO₄.7H₂O) 0.5 mM, 80 μg of natural amino acids (alanine A, cysteine C, aspartic acid D, glutamic acid B, phenylalanine F, glycine G, listidine H, isoleucine I, lysine K, leucine L, methionine M, asparagine N, proline P, glutamine Q, arginine R, serine S, threonine T, valine V, tryptophan W, tyrosine Y, 4 μg each), 250 μg of sodium dodecyl sulphate and 125 μg of β-galactosidase inducer isopropyl-β-D-thiogalactopyranoside; the mixture was then incubated at 37° C. for 75 minutes.

Enzyme-Substrate Reaction

At the end of the induction time, 132 μl of methylumbelliferyl-β-D-galactoside (MUGal) in dimethylsulfoxide (1.5 mg/ml) and 20 μl of chloroform were added to the induction mixture; after vortexing, the hydrolysis mixture was incubated for 45 minutes at 37° C. The hydrolysis was then interrupted by adding 100 μl of sodium hydroxide (NaOH) 2 N in water.

Measurement

The reading mixture was immediately transferred to a quartz cuvette and placed in the fluorimeter (Perkin-Elmer LS50B), with heating block set at 37° C.; the excitation wavelength was set at 362 nm and the emission wavelength was set at 445 nm with slit widths at 2.5 nm. A blank reading needed to be performed by simulating the sample procedure, except for the addition of cells, being potential contaminating particles absent. E. coli count was deduced from a previously calculated regression line. y=30.529x (y=cell number x=relative fluorescence units)

TABLE 1

| | r.f.u. | r.f.u. | Cells |
|---|---|---|---|
| Slit (2.5; 2.5) | (tot) | (tot-B) | (2 ml) |
| Blank B | 47 | 0 | 0 |
| Sample 1 | 84 | 37 | 1,130 |
| Sample 2 | 116 | 69 | 2,107 |
| Sample 3 | 145 | 98 | 2,992 |
| Sample 4 | 179 | 132 | 4,030 |
| Sample 5 | 211 | 164 | 5,007 |
| Sample 6 | 246 | 199 | 6,075 |
| Sample 7 | 274 | 227 | 6,930 |
| Sample 8 | 308 | 261 | 7.968 |
| Sample 9 | 325 | 278 | 8,487 |
| Sample 10 | 357 | 310 | 9,464 |
| Enterococcus 1, 2, 3 | 47 | 0 | Not det. |

Samples 1-10 relative to E. coli.
r.f.u. = relative fluorescence units.
B = Blank (background fluorescence).

### Example 2

Induction of β-Glucuronidase Activity on E. coli and Klebsiella pneumoniae and cell count when positive.

Sample Preparation

Different samples in physiological solution (sodium chloride NaCl, 0.85% in water) containing E. coli cells (ATCC 25922) and Klebsiella pneumoniae cells (ATCC 13883) were variously diluted. For each sample, the cell number was verified by plate count method.

Enzyme Induction

33 μl of sample were added to 2315 μl of filter-sterilized (0.45 μm) induction solution, as described in Example 1, except for β-glucuronidase inducer, 1.25 mg of methyl-β-D-glucuronide; the mixture was then incubated at 44° C. for 75 minutes.

Enzyme-Substrate Reaction

At the end of the induction time, 132 μl of methylumbelliferyl-β-D-glucuronide (MUGlu) in phosphate buffer (sodium hydrogen phosphate (Na₂HPO₄) 47.7 mM, potassium dihydrogen phosphate (KH₂PO₄) 22 mK magnesium sulphate heptahydrate (MgSO₄.7H2O) 0.5 mM/Triton-X 99/1 (1.5 mg/ml) and 20 μl of chloroform were added to the induction mixture; after vortexing, the hydrolysis mixture was incubated for 45 minutes at 44° C. The hydrolysis was then interrupted by adding 100 μl of sodium hydroxide (NaOH) 2 N in water.

Measurement

The reading mixture was immediately transferred to a quartz cuvette and placed in the fluorimeter Perkin-Elmer LS50B), with heating block set at 44° C.; the excitation wavelength was set at 362 mm and the emission wavelength was set at 445 nm with slit widths at 5.0 nm. A blank reading needed to be performed by simulating the sample procedure, except for the addition of cells, being potential contaminating particles absent. E. coli count was deduced from a previously calculated regression line. y=30.993x (y=cell number x=relative fluorescence units)

TABLE 2

| | r.f.u. | r.f.u. | Cells |
|---|---|---|---|
| Slit (5; 5) | (tot) | (tot-B) | (2 ml) |
| Blank B | 27 | 0 | 0 |
| Sample 1 | 84 | 14 | 434 |
| Sample 2 | 116 | 31 | 961 |
| Sample 3 | 145 | 48 | 1488 |
| Sample 4 | 179 | 61 | 1891 |
| Sample 5 | 211 | 77 | 2386 |
| Sample 6 | 246 | 89 | 2758 |
| Sample 7 | 274 | 115 | 3564 |
| Sample 8 | 308 | 127 | 3936 |
| Sample 9 | 325 | 149 | 4618 |
| Sample 10 | 357 | 147 | 4556 |
| Klebsiella 1, 2, 3 | 27 | 0 | Not det. |

Samples 1-10 relative to E. coli.
r.f.u. = relative fluorescence units.
B = Blank (background fluorescence).

### Example 3

Induction of β-Galactosidase Activity on Klebsiella pneumoniae and Cell Count When Positive.

Sample Preparation

Different samples in physiological solution (sodium chloride, 0.85% in water) containing Klebsiella pneumoniae cells (ATCC 13883) were variously diluted. Each cell number was verified by plate count method.

Enzyme Induction and Enzyme-Substrate Reaction

The enzyme induction and the expression of the enzymatic activity by cell lysis were performed according to example 1.

Measurement

The measurement was performed according to Example 1 by using a previously calculated regression line. y=60.063x (y=cell number=relative fluorescence units).

TABLE 3

| | r.f.u. | r.f.u. | Cells |
|---|---|---|---|
| Slit (5; 5) | (tot) | (tot-B) | (2 ml) |
| Blank B | 232 | 0 | 0 |
| Sample 1 | 257 | 25 | 1502 |
| Sample 2 | 305 | 73 | 4385 |
| Sample 3 | 323 | 91 | 5466 |
| Sample 4 | 329 | 97 | 5826 |
| Sample 5 | 401 | 169 | 10151 |
| Sample 6 | 427 | 195 | 11900 |
| Sample 7 | 448 | 216 | 12974 |
| Sample 8 | 523 | 291 | 17478 |
| Sample 9 | 526 | 294 | 17659 |

Samples 1-9 relative to Klebsiella pneumoniae.
r.f.u. = relative fluorescence units.
B = Blank (background fluorescence).

### Example 4

Induction of β-Galactosidase and β-Glucuronidase Activity on E. coil in Absence of Amino Acids.

Different samples in physiological solution (sodium chloride NaCl, 0.85% in water) containing E. coli cells (ATCC 25922) were variously diluted. For each sample, the cell number was verified by plate count method.

Enzyme induction, expression of the enzymatic activity by cell lysis and fluorimetric measurement were performed, except for the addition of the amino acids, according to Example 1 (β-galactosidase) and according to Example 2 (β-glucuronidase).

The results achieved from the fluorimetric assay show that, in absence of amino acids in the solution of induction, the cells do not produce the inducible enzymes in a detectable quantity.

1. An “induction solution” which provides for rapid detection of coliforms, capable to induce, in absence of cell growth, the expression of inducible enzymes β-glucuronidase and β-galactosidase, comprising:
at least one amino acid, yet preferably a mixture of amino acids, in such a quantity not to allow, between 0 and 120 minutes, a detectable cell growth of the coliforms in contact with it;
a buffer system;
bivalent ions and more particularly, the magnesium Mg⁺⁺, in a concentration preferably 0.5 mM;
an enzymatic inducer.

2. A solution according to claim 1 wherein the amino acid concentration reaches up to about 80 nM.

3. A solution according to claims 1-2 comprising at least one of the following amino acids:
tryptophan W;
at least one between methionine M and threonine T;
isoleucine I and leucine L.

4. A solution according to claim 1 wherein the amino acid is selected from the group of natural amino acids: alanine A, cysteine C, aspartic acid D, glutamic acid E, phenylalanine F, glycine G, histidine H. isoleucine I, lysine K, leucine L, methionine M, asparagine N, proline P, glutamine Q, arginine R, serine S, threonine T, valine V, tryptophan W, tyrosine Y.

5. A solution according to claims 1-4 wherein the mixture of amino acids comprises the natural amino acids in levorotatory form, ranging from about 0.01 to about 0.05 mM each.

6. A solution according to claims 1-5 wherein the buffer system is a phosphate buffer providing a pH between 6.0 and 7.5 and comprising sodium chloride (NaCl), preferably, but not necessarily, at about 0.01% (w/v).

7. A solution according to claims 1-6 wherein the enzymatic inducer is selected among isopropyl-β-D-thiogalactopyranoside for β-galactosidase, concentration being preferably about 0.2 mM, methyl-β-D-glucuronide for β-glucuronidase, concentration being preferably about 2 mM, and relative mixtures.

8. A solution according to claims 1-7 utterly comprising a selective agent acting as a membrane permeabilizer like, for instance, sodium dodecyl sulphate.

9. An analysis kit for the rapid detection of coliform cells comprising:
the induction solution A according to claims 1-8;
a solution comprising a fluorescent substrate;
instructions for use;
suitable solvents and instrumentation.

10. A kit according to claim 9 wherein the coliforms are selected among total coliforms, faecal coliforms, E. coli.

11. A kit according to claim 9 wherein the induction solution is filter-sterilized by passage through 0.45 μm or less pore size membrane filter and is in a lyophilized form.

12. A kit according to claim 9 wherein the fluorescent substrate is selected from the group consisting of methylumbelliferyl-β-D-galactoside, methylumbelliferyl-β-D-glucuronide and relative mixtures.

13. A kit according to claim 9 utterly comprising a fluorescence amplifier constituted from a solution of sodium hydroxide (NaOH) in water or any other base capable to increase pH of the induction solution to about 11-12.

14. A kit according to claim 9 utterly comprising an organic solvent, for example chloroform or any other agent capable to induce cell lysis, for example triton-x.

15. A kit according to claims 9-14 for the analysis of water samples, comprising wastewater, surface water, bathing water, freshwater, seawater, groundwater, food extracts and soil extracts.

16. A process for the rapid detection of coliform bacteria, comprising the steps of:
placing the sample to be analyzed in contact with an induction solution A, according to claims 1-8 and incubating between 30 and 50° C.;
adding a fluorogenic substrate;
rupturing the cells with any suitable lysis agent to release the induced enzyme;
assisting hydrolysis of fluorogenic substrate with discharge of the corresponding fluorophore by incubating at less than 50° C., for a period of time depending on cell number;
raising final mixture pH to about 11-12 with a basic solution, for example sodium hydroxide (NaOH) in water.

17. A process according to claim 16 wherein the temperature is between 30 and 40° C. for the detection of total coliforms and between 30 and 50° C. for the detection of faecal coliforms or E. coli.

18. A process according to claim 16 wherein a minimum time period of about 10 minutes is required for the enzymatic reaction.

19. Process according to claim 16 wherein the fluorophore concentration is determined by fluorimetric assay after extracellular hydrolysis reaction of target enzymes with relative substrates being, respectively, methylumbelliferyl-β-D-galactoside for β-galactosidase and methylumbelliferyl-β-D-glucuronide for β-glucuronidase.

20. Process according to claim 16 wherein the lysis agent is chloroform and/or triton-x.

21. Process according to claim 16 wherein the fluorescent substrate is selected from the group comprising methylumbelliferyl-β-D-galactoside in dimethylsulfoxide, methylumbelliferyl-β-D-glucuronide in water/triton-X and relative mixtures.

22. Process according to claim 16 wherein the sample to be analyzed is pre-extracted with physiological saline or phosphate buffered saline solution.

23. Process according to claim 22 wherein the sample is utterly filtered on a 0.45 μm or less membrane filter and the filter is immersed in the induction solution.

24. Process according to claim 16 wherein the sample analysis is performed by fluorimetry, being the excitation wavelength set between 330 and 390 nm and the emission wavelength set between 410 and 470 nm with slit widths between 2.5 and 20 mn.
